(19) United States
(12) Patent Application Publication
(10) Pub. No.: US 2013/0103012 A1
(43) Pub. Date: Apr. 25, 2013

(54) System and Method for Obviating Posterior Capsule Opacification

(71) Applicants: Robert Edward Grant, Laguna Beach, CA (US); David Haydn Mordaunt, Los Gatos, CA (US)

(72) Inventors: Robert Edward Grant, Laguna Beach, CA (US); David Haydn Mordaunt, Los Gatos, CA (US)

(21) Appl. No.: 13/405,137

(22) Filed: Feb. 24, 2012

Related U.S. Application Data
(60) Provisional application No. 61/550,318, filed on Oct. 21, 2011.

(51) Int. Cl. A61B 18/20

(52) U.S. Cl. 606/5

(57) ABSTRACT

A system and method are provided for obviating Posterior Capsule Opacification (PCO) which require an Optical Coherence Tomography (OCT) device for imaging the interface surface between the posterior surface of an intraocular lens (IOL) and the capsular bag. Further, the OCT device is used to identify areas of relative opacity caused by a biological growth on the interface surface in the optical zone of the IOL. A laser unit is then used to direct the focal point of a femtosecond laser beam onto the areas of relative opacity to ablate the biological growth by Laser Induced Optical Breakdown (LIOB) to thereby obviate the PCO.

This application claims the benefit of U.S. Provisional Patent Application Ser. No. 61/550,318, filed Oct. 21, 2011.

### FIELD OF THE INVENTION

The present invention pertains generally to laser systems and methods for performing ophthalmic surgical procedures. More particularly, the present invention pertains to postoperative follow-up procedures that rectify adverse consequences of a prior surgical procedure. The present invention is particularly, but not exclusively, useful as a system and method for removing biological growth from the optical zone at the posterior of an Intraocular Lens (IOL) that would otherwise cause what is generally known as Posterior Capsule Opacification (PCO).

### BACKGROUND OF THE INVENTION

After a lensectomy, wherein a cataract lens is removed from its capsular bag, it will happen that the capsular bag shrinks onto the Intraocular Lens (IOL) that has been used to replace the cataract lens. An undesirable consequence that can happen along with this postoperative shrinkage of the capsular bag is a condition known as Posterior Capsule Opacification (PCO). Optically, PCO manifests itself as visual disturbances, such as glare and gradual vision loss. In this process, a significant contributing factor for PCO is the migration (growth) of epithelial cells toward the posterior capsule. In the event, PCO is particularly problematic when opacification occurs in the optical zone of the IOL.

Heretofore, a procedure for obviating the adverse effects of PCO has been to employ a Neodymium-doped Yttrium Aluminum Garnet (Nd:YAG) laser to remove the material that is causing PCO. One approach for this procedure has been to perform a capsulotomy wherein the posterior capsule is fenestrated, and PCO material is removed through the resultant holes in the capsule. In this procedure, however, Nd:YAG lasers will typically cause small disturbances at the beam's focal point during such a procedure. In the context of PCO, these disturbances, though possibly minor, are known to be of sufficient magnitude to actually move an IOL from its position in a capsular bag. In turn, this can cause the optical characteristics of the IOL to become misaligned. This is to be avoided.

Optical Coherence Tomography (OCT) is a known imaging technique that can be effectively used to create three dimensional images inside an eye. Of particular interest here is the interface region between the posterior surface of an IOL and the capsular bag. In addition to imaging such an interface, OCT techniques can also be used to determine the intensity of the light that is being reflected from an object, such as the biological growths that cause PCO at the interface between the posterior surface of an IOL and the capsular bag. Thus, based on these capabilities, it is apparent that OCT techniques are capable of providing valuable information about the postoperative progression and extent of PCO.

Another relatively recent technical development has been the use of femtosecond laser units for the ablation of tissue and other cellular structures. In particular, it is known that such ablation can be effectively accomplished by a process known as Laser Induced Optical Breakdown (LIOB). Importantly, femtosecond laser beams are capable of photoablating tissue by LIOB with extreme precision (e.g. to within tolerances of 10-50 microns), at very low energy levels (e.g. below 50 micro joules). Stated differently, the fluence level of a femtosecond laser beam and the location of its focal point can be controlled with great accuracy. In the context of PCO, it is also envisioned that femtosecond lasers can create a disruptive laser-acoustic-mechanical effect on PCO material that will beneficially contribute to the ablation of this material by LIOB.

In light of the above, it is an object of the present invention to provide a system and method for obviating the adverse effects of PCO. Another object of the present invention is to provide a system and method that uses OCT imaging techniques to locate areas of PCO on an IOL, and to then use OCT imaging techniques to control a laser unit for the LIOB and/or laser-acoustic-mechanical disruption of the biological growths that are causing the PCO. Yet another object of the present invention is to provide a system and method for obviating the adverse effects of PCO which avoid disturbances against the IOL that would otherwise cause the IOL to become optically misaligned. Still another object of the present invention is to provide a system and corresponding method for obviating PCO that is easy to use, is simple to manufacture and is comparatively cost effective.

### SUMMARY OF THE INVENTION

In accordance with the present invention a system and method are provided for obviating Posterior Capsule Opacification (PCO). For the present invention, this is done by using Laser Induced Optical Breakdown (LIOB) techniques to ablate the biological growth that sometimes forms on the posterior surface of an Intraocular Lens (IOL). As a complement to LIOB, in conjunction therewith, or as an alternative procedure, the present invention also envisions disrupting PCO material in response to a laser-acoustic-mechanical effect. In any event, the opacification of interest for the present invention occurs in the optical zone of the IOL subsequent to a lensectomy.

Structurally, the system of the present invention includes a laser unit for both generating a femtosecond laser beam, and for focusing the laser beam to a focal point for the LIOB of a biological growth. Preferably, the laser beam is a pulsed laser beam, and each pulse in the laser beam will have an energy level that is less than approximately fifty micro joules (<50 μJ). Further, each pulse preferably has a pulse duration that is less than approximately 500 femtoseconds. Importantly, the laser beam is configured to ablate biological growth that can form on the posterior surface of an IOL after the IOL has been implanted into a capsular bag.

In addition to the laser unit, the system of the present invention also includes an imaging unit. Preferably, the imaging unit is an Optical Coherence Tomography (OCT) device that is capable of creating a three dimensional image of an interface surface in situ, inside an eye. In particular, as indicated above, for purposes of the present invention, this interface surface will lie between the posterior surface of an intraocular lens (IOL) and the capsular bag in which the IOL has been implanted.

An analyzer that is connected as an operational component of the imaging unit is provided for identifying at least one area of relative opacity on the interface surface. Of particular importance here, are areas in the optical zone of the IOL where increased opacity has been caused by a biological growth at the interface surface.

Operationally, a computer is used for controlling the laser unit. In particular, this is done to direct the focal point of the laser beam onto defined regions in areas of relative opacity, and to then move the focal point of the femtosecond laser beam over these areas of opacity to ablate the biological growth. More specifically, in order to avoid any potential damage to the IOL during an LIOB ablation or laser-acoustic-mechanical disruption of PCO material, the focal point is appropriately distanced from the IOL. In this process, a monitor that is connected to the analyzer measures a reflectivity value for the light that is reflected from an area of relative opacity. A comparator, that is connected to both the monitor and the computer, then compares the reflectivity value that is received from the monitor with a base reflectivity value. Specifically, this comparison establishes a reflectivity differential which can be used by the computer to identify areas of opacity and to cease directing the focal point of the laser beam toward a defined region in the area of relative opacity when the reflectivity differential is effectively a null in the defined region.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic presentation of the functional components in a system for obviating PCO in accordance with the present invention; and

FIG. 2 is a cross sectional view of the interface between the posterior of an IOL and the capsular bag, as seen along the line 2-2 in FIG. 1, along with exemplary light beams to illustrate the reflective consequences of PCO.

### DESCRIPTION OF THE PREFERRED EMBODIMENTS

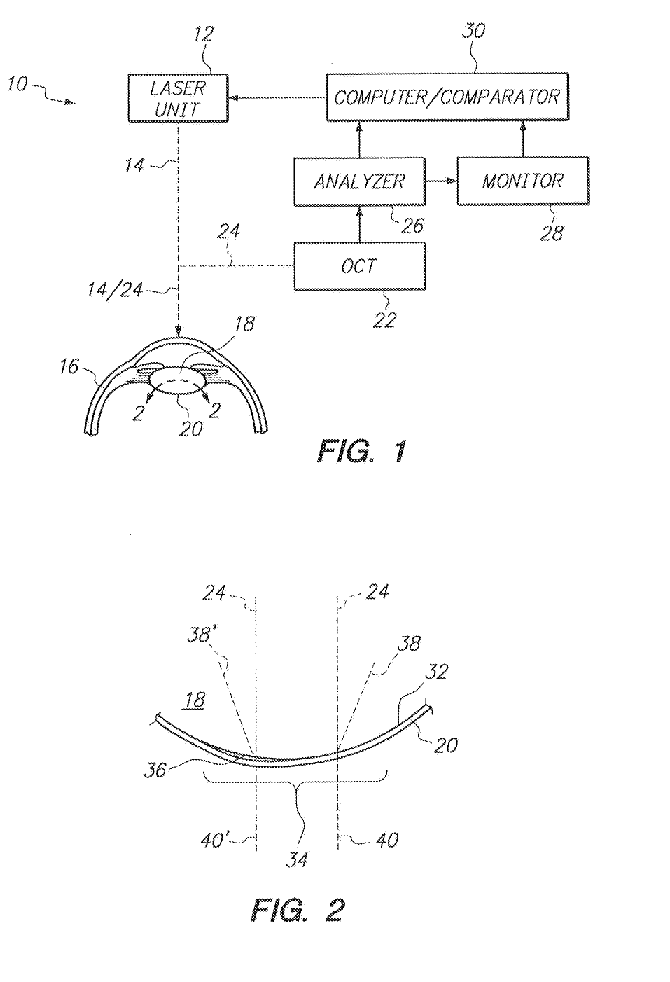

Referring initially to FIG. 1 a system for obviating Posterior Capsule Opacification (PCO) in accordance with the present invention is shown and is generally designated 10. As shown, the system 10 includes a laser unit 12 that will generate a laser beam 14, and direct the laser beam 14 toward an eye 16. More specifically, the laser beam 14 is directed by the laser unit 12 to a focal point between the posterior of an Intraocular Lens (IOL) 18 and the capsular bag 20 of the eye 16 wherein the IOL 18 has been implanted. For purposes of the present invention the laser beam 14 is preferably a pulsed laser beam 14 and each pulse of the laser beam 14 has an energy level that is less than approximately fifty micro joules (<50 μJ). Further, each pulse of the laser beam 14 has a duration that is approximately 500 femtoseconds.

FIG. 1 also shows that the system 10 includes an imaging unit 22 that is preferably an Optical Coherence Tomography (OCT) device. Importantly, this imaging unit 22 needs to be capable of using an imaging beam 24 to create three dimensional images of tissue and structures inside the eye 16. In FIG. 1 the OCT imaging unit 22 is shown connected to an analyzer 26 which, in turn, is connected to a monitor 28. As shown, both the analyzer 26 and the monitor 28 are connected to a computer/comparator 30. With these connections, the system 10 is capable of providing input to the computer/comparator 30 that includes information which is contained in images created by the OCT imaging unit 22. Of particular interest here is in situ information about an interface surface 32 that lies between the IOL 18 and the capsular bag 20.

In FIG. 2 it is shown that as the imaging beam 24 passes through the eye 16 it will also pass through the interface surface 32 between the IOL 18 and the capsular bag 20, and an optical zone 34 of the IOL 18. Of particular importance here is that the optical zone 34 of the IOL 18 is indicative of the part of IOL 18 that must be transparent in order for the IOL 18 to be optically effective. With this in mind, FIG. 2 also indicates that a biological growth 36 can sometimes form over the interface surface 32 in the optical zone 34. This biological growth 36 will then create an opacity that can be detrimental to the visual capabilities of the eye 16 (i.e. the biological growth 36 will cause the condition known as Posterior Capsule Opacity (PCO)). Thus, the biological growth 36 needs to be removed. As envisioned for the system 10, this removal is accomplished by the laser unit 12 using LIOB techniques.

Still referring to FIG. 2, it will be appreciated that in those areas of the optical zone 34 where there is no biological growth 36, the imaging beam 24 will be generally unaffected. In this situation, the light 38 that is reflected/scattered from the interface surface 32 will be minimal in relation to the light 40 that transits the interface surface 32. Stated differently, the vast preponderance of light in the imaging beam 24 will pass through the IOL 18 and the capsular bag 20, and emerge as transient light 40. Thus, the reflected/scattered light 38 will have quite a low reflectivity value. As used here, reflectivity value is a ratio of the intensity of reflected/scattered light beam 38 to the incident imaging beam 24. In this case, the ratio will be much less than one. The situation is quite different, however, when light of the imaging beam 24 is incident on any biological growth 36 that is located at the interface surface 32. In this latter case, the light 38′, that is reflected/scattered from the biological growth 36, will have an intensity that is nearer to that of the incident imaging beam 24. Stated differently, a greater amount of light in the imaging beam 24 will not transit through biological growth 36 at the interface surface 32 (i.e. transient light 40′ will be minimal). Thus, the reflected/scattered light 38′ will have a relatively high reflectivity value (i.e. the ratio of reflected/scattered light beam 38 to the incident imaging beam 24 will tend toward a value of one).

As intended for the system 10, the reflectivity value of reflected/scattered light 38 or 38′ is determined by the analyzer 26. This reflectivity value is then compared with a base reference that is established by the computer/comparator 30 to measure a reflectivity differential. This reflectivity differential is then used by the computer/comparator 30 to cease using the laser beam 14 for the LIOB of biological growth 36 when the reflectivity differential is effectively a null.

A methodology for using the system 10 includes a first step of imaging the interface surface 32, in situ, using the OCT imaging unit 22. During this imaging, a reflectivity value for light that is reflected from defined areas of the interface surface 32 is measured by the analyzer 26. The various reflectivity values are then compared with a base reference to establish respective reflectivity differentials. Next, based on the various reflectivity differentials, the analyzer 26 identifies areas of relative opacity on the interface surface 32 that are caused by a biological growth 36. The laser unit 12 then directs the focal point of the femtosecond laser beam 14 to ablate the biological growth 36 by Laser Induced Optical Breakdown (LIOB). In addition to the ablation of biological growth 36 by LIOB, the focal point of laser beam 14 can be used to disrupt the biological growth 36 by a phenomenon referred to herein as the laser-acoustic-mechanical effect. As envisioned for the present invention, the laser-acoustic-mechanical effect will be used to either facilitate LIOB, or to complement LIOB as a means for removing the biological growth 36. In any event, during the ablation of biological growth 36, the reflectivity differential is measured by the computer/comparator 30 and used to cease directing the focal point of the laser beam 14 toward a defined region in the area of relative opacity when the reflectivity differential is effectively a null in the defined region.

While the particular System and Method for Obviating Posterior Capsule Opacification as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for obviating Posterior Capsule Opacification (PCO) which comprises the steps of:
imaging an interface surface in situ, wherein the interface surface lies between the posterior surface of an intraocular lens (IOL) and a capsular bag;
identifying at least one area of relative opacity on the interface surface, wherein the relative opacity is caused by a biological growth at the interface surface;
directing the focal point of a femtosecond laser beam onto the area of relative opacity; and
moving the focal point of the femtosecond laser beam over the area to remove the biological growth.

2. A method as recited in claim 1 further comprising the steps of:
measuring a reflectivity value of light reflected from the area of relative opacity; and
comparing the reflectivity value received from the monitor with a base reference to establish a reflectivity differential therebetween, wherein the reflectivity differential is used by the computer to cease directing the focal point of the laser beam toward a defined region in the area of relative opacity when the reflectivity differential is effectively a null in the defined region.

3. A method as recited in claim 1 wherein the removal of biological growth in the moving step is accomplished by Laser Induced Optical Breakdown (LIOB), wherein the laser beam is a pulsed beam, and each pulse of the laser beam has an energy level less than approximately fifty micro joules (<50 μJ) and a duration of approximately 500 femtoseconds.

4. A method as recited in claim 1 wherein the removal of biological growth in the moving step is accomplished by a laser-acoustic-mechanical effect.

5. A method as recited in claim 1 wherein the imaging step is accomplished using an Optical Coherence Tomography (OCT) device.

6. A method as recited in claim 1 wherein the directing step is controlled by a computer, and wherein the computer is responsive to a three dimensional image of the area.

7. A method as recited in claim 6 wherein the area is in an optical zone of the IOL.

8. A system for obviating Posterior Capsule Opacification (PCO) which comprises:
a laser unit for generating a femtosecond laser beam and for focusing the laser beam to a focal point;
an imaging unit for creating an image of an interface surface in situ, wherein the interface surface lies between the posterior surface of an intraocular lens (IOL) and a capsular bag;
an analyzer connected to the imaging unit for identifying at least one area of relative opacity on the interface surface, wherein the relative opacity is caused by a biological growth at the interface surface; and
a computer for controlling the laser unit to direct the focal point of the laser beam onto defined regions in the area of relative opacity, and for moving the focal point of the femtosecond laser beam over the area to ablate the biological growth.

9. A system as recited in claim 8 further comprising:
a monitor connected to the analyzer for measuring a reflectivity value of light reflected from the area of relative opacity; and
a comparator connected to the monitor and to the computer for comparing the reflectivity value received from the monitor with a base reference to establish a reflectivity differential therebetween, wherein the reflectivity differential is used by the computer to cease directing the focal point of the laser beam toward a defined region in the area of relative opacity when the reflectivity differential is effectively a null in the defined region.

10. A system as recited in claim 8 wherein the laser beam is a pulsed beam, and each pulse of the laser beam has an energy level less than approximately fifty micro joules (<50 μJ) and each pulse has a duration of approximately 500 femtoseconds.

11. A system as recited in claim 8 wherein the area is in the optical zone of the IOL.

12. A system as recited in claim 8 wherein the imaging unit employs Optical Coherence Tomography (OCT) techniques.

13. A system as recited in claim 8 wherein ablation of the biological tissue is accomplished by Laser Induced Optical Breakdown (LIOB).

14. A method for obviating Posterior Capsule Opacification (PCO) which comprises the steps of:
imaging an interface surface in situ, wherein the interface surface lies between the posterior surface of an intraocular lens (IOL) and a capsular bag;
identifying at least one area of relative opacity on the interface surface, wherein the relative opacity is caused by a biological growth at the interface surface;
measuring a reflectivity value of light reflected from the area of relative opacity;
generating a femtosecond laser beam;
focusing the laser beam to a focal point;
directing the focal point of the laser beam onto defined regions in the area of relative opacity;
moving the focal point of the femtosecond laser beam over the area of relative opacity to ablate the biological growth by Laser Induced Optical Breakdown (LIOB);
comparing a reflectivity value received from the area of relative opacity in response to the moving step, with a base reference, to establish a reflectivity differential therebetween; and
using the reflectivity differential obtained in the comparing step to cease directing the focal point of the laser beam toward a defined region in the area of relative opacity when the reflectivity differential in the defined region is effectively a null.

15. A method as recited in claim 14 wherein the femtosecond laser beam is pulsed, with each pulse having an energy level and a duration, and wherein the method further comprises the step of correlating the energy level and the duration of the pulse.

16. A method as recited in claim 15 wherein the energy level is less than approximately fifty micro joules (<50 μJ) and the duration is approximately 500 femtoseconds.

17. A method as recited in claim 14 wherein the base reflectivity value is less than the received reflectivity value.

18. A method as recited in claim 14 wherein the imaging step, the identifying step and the measuring step are accomplished using Optical Coherence Tomography (OCT) techniques.

19. A computer program product for obviating Posterior Capsule Opacification (PCO), wherein the computer program product comprises program sections for respectively: generating a femtosecond laser beam and for focusing the laser beam to a focal point; creating an image of an interface surface in situ, wherein the interface surface lies between the posterior surface of an intraocular lens (IOL) and a capsular bag; identifying at least one area of relative opacity on the interface surface, wherein the relative opacity is caused by a biological growth at the interface surface; and controlling the laser unit to direct the focal point of the laser beam onto defined regions in the area of relative opacity, and for moving the focal point of the femtosecond laser beam over the area to ablate the biological growth.

20. A computer program product as recited in claim 19 further comprising program sections for: measuring a reflectivity value of light reflected from the area of relative opacity; and comparing the reflectivity value received from the monitor with a base reference to establish a reflectivity differential therebetween, wherein the reflectivity differential is used by the computer to cease directing the focal point of the laser beam toward a defined region in the area of relative opacity when the reflectivity differential is effectively a null in the defined region.
